(19) United States
(12) Patent Application Publication
(10) Pub. No.: US 2002/0064808 A1
(43) Pub. Date: May 30, 2002

(54) Focused acoustic energy for ejecting cells from a fluid

(76) Inventors: Mitchell W. Mutz, Palo Alto, CA (US); Richard N. Ellson, Palo Alto, CA (US)

Correspondence Address: Dianne E. Reed, REED & ASSOCIATES, 3282 Alpine Road, Portola Valley, CA, 94028, US

(21) Appl. No.: 09/727,391

(22) Filed: Nov. 29, 2000

(51) Int. Cl. C12Q 1/02; C12N 15/01

(52) U.S. Cl. 435/40.5; 435/446

(57) ABSTRACT

This invention is directed to the use of focused acoustic energy in the spatially directed ejection of cells suspended in a carrier fluid, for printing and patterning cells onto a substrate surface, for example to pattern an array of cells onto a substrate. An array of cells on a substrate surface comprising an array of substantially planar sites, with each site containing a single cell, is consequently also provided. Also disclosed are methods for the systematic generation and screening of arrays of living cells on a substrate from fluids containing one or more living cells. A method of attaching cells displaying a specific marker moiety on their surface, through specific recognition of the marker moiety by a cognate moiety that is linked to the surface is provided. Cells may be transformed to display a specific marker recognized by a corresponding cognate moiety, or the marker moiety may appear on untransformed cells. Cells displaying marker moieties may be conveniently attached to a surface functionalized with the cognate moiety. The combination of acoustic ejection and the marker moiety/cognate moiety system can be employed to select cells displaying a specific marker for adhesion to a substrate surface. The combination of several different marker moieties uniquely displayed on the cell surface of different types of cells combined with an array of different cognate moieties on a substrate may be employed in conjunction with the acoustic ejection of single living cells to create cell arrays with a specific number of cells displaying a specific marker attached to the substrate surface at a desired locale or region.

### TECHNICAL FIELD

This invention relates generally to the use of focused acoustic energy in the spatially directed ejection of cells suspended in a carrier fluid, for printing and patterning cells onto a substrate surface, for example to pattern an array of cells onto a substrate.

### BACKGROUND

Arrays of single living cells have been made by inserting individual cells into individual well sites or holes that are open on both the top and bottom, with the top opening large enough for the desired cell to pass through and the bottom opening too small for the desired cell to pass through (Weinreb et al., U.S. Pat. No. 5,506,141). Microfabrication techniques for manufacturing arrays of such well sites or holes are well known, as the diameters of eukaryotic cells are larger than about 10 μm and the smallest prokaryotic cells, genus Mycoplasma, are about 0.15-30 μm or larger (for example, Chu et al. in U.S. Pat No. 6,044,981 teach methods for making holes or channels having dimensions as small as about 5 nanometers by employing a sacrificial layer, these dimensions are smaller than the resolution limit of photolithography, currently 0.35 μm). There are no methods of manipulating cells currently employed which permit making an ordered array of single cells at different locations of a planar substrate surface. Further, no methods of separating cells into individual array sites by size exists other than by controlling physical hole or well size as described by Weinreb et al., supra, to permit cell populations of differing size to enter and be contained in non-planar holes or wells. Furthermore, no methods for containing individual cells to array sites other than by utilization of non-planar holes and wells of appropriate size.

Although the screening of cells is appreciated to initially require a relatively large known number of individual cells (as described for example by Weinreb et al., U.S. Pat. No. 5,506,141) to ensure detection of a particular cell function or characteristic among a population of cells at different life cycle stages and having other variations between individual cells, simultaneous delivery of screening and other reagents requires fluidic nexus between each single cell container. Taylor, U.S. Pat. No. 6,103,479 describes a miniaturized cell array method and device for screening cells comprising cells in physical wells that are microfluidically connected to independent reagent sources by microchannels which can supply fluid reagents to individual or multiple cells arrayed in the physical wells. Such systems may be easily altered to permit tests on individual cells or a large group simultaneously, but require costly and detailed microfabrication. The site density of such arrays is limited by the need to make individual wells with physical requirements such as minimum well wall thickness for physical integrity and additional space for the channels themselves. Thus a need exists for maximizing the site density while maintaining flexibility for assaying populations and subpopulations and reducing microfabrication time, expense and cost. Further a need exists for microfluidic delivery of reagents to arrayed cells, whether or not contained in physical wells or localized on a planar substrate in virtual wells, without requiring a corresponding array os individual microfabricated channels for supplying each site with a desired reagent.

No method or device is known to exist for manipulating individual cells by ejecting them from a fluid onto a substrate surface without killing the cells. Thus a need exists for a method and corresponding device for ejecting a single cell from a fluid to a chosen surface locale or region to permit selective ejection for patterning of cells on a surface for making arrays and other applications requiring cell pattering on a surface, such as engineering tissues and the like, or simply for sorting cells.

### SUMMARY OF THE INVENTION

Accordingly, it is an object of the present invention to provide devices and methods that overcome the above-mentioned disadvantages of the prior art.

In one aspect of the invention, a method is provided for acoustically ejecting a plurality of single cells contained in fluid droplets toward designated sites on a substrate surface for deposition on the substrate surface using a device substantially as described in U.S. patent application Ser. No. 09/669,996 (“Acoustic Ejection of Fluids from a Plurality of Reservoirs”), inventors Ellson, Foote and Mutz, filed on Sep. 25, 2000, and assigned to Picoliter, Inc. (Cupertino, Calif.). As described in the aforementioned patent application, the device enables acoustic ejection of a plurality of fluid droplets toward designated sites on a substrate surface for deposition thereon, and: a plurality of cell containers or reservoirs each adapted to contain a fluid capable of carrying, for example, cells suspended therein; an acoustic ejector for generating acoustic radiation and a focusing means for focusing it at a focal point near the fluid surface in each of the reservoirs; and a means for positioning the ejector in acoustic coupling relationship to each of the cell containers or reservoirs. Preferably, each of the containers is removable, comprised of an individual well in a well plate, and/or arranged in an array. The cell containers or reservoirs are preferably also substantially acoustically indistinguishable from one another, have appropriate acoustic impedance to allow the energetically efficient focusing of acoustic energy near the surface of a contained fluid, and are capable of withstanding conditions of the fluid-containing reagent.

In another aspect of the invention, an array of cells is provided on a substrate surface comprising an array of substantially planar sites, wherein each site contains a single cell. The array is prepared by positioning an acoustic ejector so as to be in acoustically coupled relationship with a first carrier fluid cell suspension-containing reservoir containing a first carrier fluid and suspension of one cell type or clone, or a mixture of cell types or clones. After acoustic detection of the presence of a cell sufficiently close to the fluid surface, and detection of any properties used as criteria for ejection, the ejector is activated to generate and direct acoustic radiation so as to have a focal point within the carrier fluid and near the surface thereof and an energy sufficient to eject a droplet of carrier fluid having a volume capable of containing a single cell, thereby ejecting a single cell contained in fluid droplet toward a first designated site on the substrate surface. Additional cells may be ejected from the first container. Or, the ejector may be repositioned so as to be in acoustically coupled relationship with a second carrier fluid cell suspension-containing reservoir and the process is repeated as above to eject a single cell contained in droplet of the second fluid toward a second designated site on the substrate surface, wherein the first and second designated sites may or may not be the same. If desired, the method may be repeated with a plurality of cells from each container, with each reservoir generally although not necessarily containing a suspension of different cells or cell mixtures. The acoustic ejector is thus repeatedly repositioned so as to eject a single cell containing droplet from each reservoir toward a different designated site on a substrate surface. In such a way, the method is readily adapted for use in generating an array of cell on a substrate surface. The arrayed cells may be attached to the substrate surface by one or more external marker moiety cognate moiety specific binding system, an example of one such specific binding system being streptavidin as an external marker, effected by transformation with the cognate moiety being biotin, multiple specific binding systems include externally displayed IgM clones and epitopes as the cognate moiety.

In another aspect, the invention relates to a method for ejecting fluids from fluid reservoirs toward designated sites on a substrate surface where live cells reside for cell screening. This aspect of the invention relates to a method for the systematic screening of cell arrays by channel-less microfluidic delivery by acoustic ejection for selective screening of desired sites, parallel screening of all sites simultaneously effected by immersion of the whole array in a reagent. In another aspect of the invention a system for making, and screening and characterizing live cell arrays is provided.

In yet another aspect, the invention provides a method of forming arrays of single live cells more rapidly flexibly and economically than by approaches requiring use of holes or physical wells and independent channel based microfluidic delivery.

Yet another aspect of the invention provides relatively high density arrays of live cells, e.g. higher density than attainable by approaches requiring use of holes or physical wells and independent channel based microfluidic delivery.

Yet another aspect of the invention is ejection of selected live cells from a fluid.

A final aspect of the invention is the general spatial patterning of cells on a surface with or without a specific attachment system.

### BRIEF DESCRIPTION OF THE DRAWINGS

FIGS. 1A and 1B, collectively referred to as FIG. 1, schematically illustrate in simplified cross-sectional view an embodiment of a device useful in conjunction with the invention, the device comprising first and second cell containers or reservoirs, an acoustic ejector, and an ejector positioning means. FIG. 1A shows the acoustic ejector acoustically coupled to the first cell container or reservoir and having been activated in order to eject a droplet of fluid containing a single cell from within the first cell container or reservoir toward a designated site on a substrate surface. FIG. 1B shows the acoustic ejector acoustically coupled to a second cell container or reservoir.

FIGS. 2A, 2B and 2C, collectively referred to as FIG. 2, illustrate in schematic view a variation of the inventive embodiment of FIG. 1 wherein the cell containers or reservoirs comprise individual wells in a reservoir well plate and the substrate comprises a smaller well plate with a corresponding number of wells. FIG. 2A is a schematic top plane view of the two well plates, i.e., the cell container or reservoir well plate and the substrate surface having arrayed cells contained in fluid droplets. FIG. 2B illustrates in cross-sectional view a device comprising the cell container or reservoir well plate of FIG. 2A acoustically coupled to an acoustic ejector, wherein a cell contained in a droplet is ejected from a first well of the cell container or reservoir well plate into a first well of the substrate well plate. FIG. 2C illustrates in cross-sectional view the device illustrated in FIG. 2B, wherein the acoustic ejector is acoustically coupled to a second well of the cell container or reservoir well plate and further wherein the device is aligned to enable the acoustic ejector to eject a droplet from the second well of the cell container or reservoir well plate to a second well of the substrate well plate.

FIGS. 3A, 3B, 3C and 3D, collectively referred to as FIG. 3, schematically illustrate in simplified cross-sectional view an embodiment of the inventive method in which cells having an externally displayed marker moiety are ejected onto a substrate using the device of FIG. 1. FIG. 3A illustrates the ejection of a cell containing fluid droplet onto a designated site of a substrate surface. FIG. 3B illustrates the ejection of a droplet containing a first cell displaying a first marker moiety adapted for attachment to a modified substrate surface to which a first. FIG. 3C illustrates the ejection of a droplet of second fluid containing a second molecular moiety adapted for attachment to the first molecule. FIG. 3D illustrates the substrate and the dimer synthesized in situ by the process illustrated in FIGS. 3A, 3B and 3C.

FIGS. 4A and 4B, collectively referred to as FIG. 4, depict arrayed cells contained in droplets deposited by acoustic ejection using the device of FIG. 1. FIG. 4A illustrates two different cells resident at adjacent array sites, contained in fluid droplets adhering to a designated site of a substrate surface by surface tension, with each cell further attached to the site by binding of streptavidin (SA) to a biotinylated (biotin (B) linked) surface. Streptavidin is displayed on the cell exterior as a result of transformation by an external display targeted streptavidin coding sequence containing construct. FIG. 4B illustrates two different cells resident at adjacent array sites, contained in fluid droplets adhering to a designated site of a substrate surface by surface tension, with each cell further attached to the site by binding of an externally displayed antigenic epitope characteristic to the cell (here E1 and E2) to a two different monoclonal antibodies (mAb-E1, mAb-E2) specific respectively for the different epitopes, each mAb linked to the surface at only one of the adjacent array sites.

FIGS. 5A, 5B and 5C, collectively referred to as FIG. 5, depict a device having a fluidic channel as the container from which the cells are ejected onto the substrate. FIG. 5A and FIG. 5B illustrate the device as a schematic. 5C illustrates top view of channels containing live cells the substrate surface having arrayed cells contained in fluid droplets. FIG. 5D illustrates cross section of channel showing physical upwards protrusion of channel floor to direct cells to sufficiently close to fluid surface for ejection. FIG. 5E illustrates cross section of channel showing use of focused energy, such as acoustic energy, to direct cells to sufficiently close to fluid surface for ejection.

### DETAILED DESCRIPTION OF THE INVENTION

Before describing the present invention in detail, it is to be understood that this invention is not limited to specific fluids, biomolecules or device structures, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a cell container” or “a reservoir” includes a plurality of cell containers or reservoirs, reference to a fluid ” includes a plurality of fluids, reference to “a biomolecule” includes a combination of biomolecules, and the like.

In describing and claiming the present invention, the following terminology will be used in accordance with the definitions set out below.

The terms “acoustic coupling” and “acoustically coupled” used herein refer to a state wherein an object is placed in direct or indirect contact with another object so as to allow acoustic radiation to be transferred between the objects without substantial loss of acoustic energy. When two items are indirectly acoustically coupled, an “acoustic coupling medium” is needed to provide an intermediary through which acoustic radiation may be transmitted. Thus, an ejector may be acoustically coupled to a fluid, e.g., by immersing the ejector in the fluid or by interposing an acoustic coupling medium between the ejector and the fluid to transfer acoustic radiation generated by the ejector through the acoustic coupling medium and into the fluid.

The term “adsorb” as used herein refers to the noncovalent retention of a molecule by a substrate surface. That is, adsorption occurs as a result of noncovalent interaction between a substrate surface and adsorbing moieties present on the molecule that is adsorbed. Adsorption may occur through hydrogen bonding, van der Waal's forces, polar attraction or electrostatic forces (i.e., through ionic bonding). Examples of adsorbing moieties include, but are not limited to, amine groups, carboxylic acid moieties, hydroxyl groups, nitroso groups, sulfones and the like. Often the substrate may be functionalized with adsorbent moieties to interact in a certain manner, as when the surface is functionalized with amino groups to render it positively charged in a pH neutral aqueous environment. Likewise, adsorbate moieties may be added in some cases to effect adsorption, as when a basic protein is fused with an acidic peptide sequence to render adsorbate moieties that can interact electrostatically with a positively charged adsorbent moiety.

The term “array” used herein refers to a two-dimensional arrangement of features such as an arrangement of reservoirs (e.g., wells in a well plate) or an arrangement of different materials including ionic, metallic or covalent crystalline, including molecular crystalline, composite or ceramic, glassine, amorphous, fluidic or molecular materials on a substrate surface (as in an oligonucleotide or peptidic array). Different materials in the context of molecular materials includes chemical isomers, including constitutional, geometric and stereoisomers, and in the context of polymeric molecules constitutional isomers having different monomer sequences. Arrays are generally comprised of regular, ordered features, as in, for example, a rectilinear grid, parallel stripes, spirals, and the like, but non-ordered arrays also may be used. An array is distinguished from the more general term pattern in that patterns do not necessarily contain regular and ordered features. The arrays or patterns formed using the devices and methods of the invention have no optical significance to the unaided human eye. For example, the invention does not involve ink printing on paper or other substrates in order to form letters, numbers, bar codes, figures, or other inscriptions that have visual significance to the unaided human eye. In addition, arrays and patterns formed by the deposition of ejected droplets on a surface as provided herein are preferably substantially invisible to the unaided human eye. Arrays typically but do not necessarily comprise at least about 4 to about 10,000,000 features, generally in the range of about 4 to about 1,000,000 features.

The term “attached,” as in, for example, a substrate surface having a molecular moiety “attached” thereto (e.g., in the individual molecular moieties in arrays generated using the methodology of the invention) includes covalent binding, adsorption, and physical immobilization. The terms “binding” and “bound” are identical in meaning to the term “attached.”

The term “biomolecule” as used herein refers to any organic molecule, whether naturally occurring, recombinantly produced, or chemically synthesized in whole or in part, that is, was or can be a part of a living organism, or synthetic analogs of molecules occurring in living organisms including nucleic acid analogs having peptide backbones and purine and pyrimidine sequence, carbamate backbones having side chain sequence resembling peptide sequences, and analogs of biological molecules such as epinephrine, GABA, endorphins, interleukins and steroids. The term encompasses, for example, nucleotides, amino acids and monosaccharides, as well as oligomeric and polymeric species such as oligonucleotides and polynucleotides, peptidic molecules such as oligopeptides, polypeptides and proteins, saccharides such as disaccharides, oligosaccharides, polysaccharides, mucopolysaccharides or peptidoglycans (peptido-polysaccharides) and the like. The term also encompasses synthetic GABA analogs such as benzodiazepines, synthetic epinephrine analogs such as isoproterenol and albuterol, synthetic glucocorticoids such as prednisone and betamethasone, and synthetic combinations of naturally occurring biomolecules with synthetic biomolecules, such as theophylline covalently linked to betamethasone.

It will be appreciated that, as used herein, the terms “nucleoside” and “nucleotide” refer to nucleosides and nucleotides containing not only the conventional purine and pyrimidine bases, i.e., adenine (A), thymine (T), cytosine (C), guanine (G) and uracil (U), but also protected forms thereof, e.g., wherein the base is protected with a protecting group such as acetyl, difluoroacetyl, trifluoroacetyl, isobutyryl or benzoyl, and purine and pyrimidine analogs. Suitable analogs will be known to those skilled in the art and are described in the pertinent texts and literature. Common analogs include, but are not limited to, 1-methyladenine, 2-methyladenine, N⁶-methyladenine, N⁶-isopentyladenine, 2-methylthio-N⁶-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “oligonucleotide” shall be generic to polydeoxynucleotides (containing 2-deoxy-D-ribose), to polyribonucleotides (containing D-ribose), to any other type of polynucleotide which is an N-glycoside of a purine or pyrimidine base, and to other polymers containing nonnucleotidic backbones (for example PNAs), providing that the polymers contain nucleobases in a configuration that allows for base pairing and base stacking, such as is found in DNA and RNA. Thus, these terms include known types of oligonucleotide modifications, for example, substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.).

There is no intended distinction in length between the term “polynucleotide” and “oligonucleotide,” and these terms will be used interchangeably. These terms refer only to the primary structure of the molecule. As used herein the symbols for nucleotides and polynucleotides are according to the IUPAC-IUB Commission of Biochemical Nomenclature recommendations (Biochemistry 9:4022, 1970).

“Peptidic” molecules refer to peptides, peptide fragments, and proteins, i.e., oligomers or polymers wherein the constituent monomers are alpha amino acids linked through amide bonds. The amino acids of the peptidic molecules herein include the twenty conventional amino acids, stereoisomers (e.g., D-amino acids) of the conventional amino acids, unnatural amino acids such as α,α-disubstituted amino acids, N-alkyl amino acids, and other unconventional amino acids. Examples of unconventional amino acids include, but are not limited to, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine.

The term “fluid” as used herein refers to matter that is nonsolid or at least partially gaseous and/or liquid. A fluid may contain a solid that is minimally, partially or fully solvated, dispersed or suspended; particles comprised of gels or discrete fluids may also be suspended in a fluid. Examples of fluids include, without limitation, aqueous liquids (including water per se and salt water) and nonaqueous liquids such as organic solvents and the like. live cells suspended in a carrier fluid is an example of a gel or discrete fluid suspended in a fluid. As used herein, the term “fluid” is not synonymous with the term “ink” in that an ink must contain a colorant and may not be gaseous and/or liquid.

The term “acoustic focusing means” as used herein refers to causing acoustic waves to converge at a focal point by either a device separate from the acoustic energy source that acts like an optical lens, or by the spatial arrangement of acoustic energy sources to effect convergence of acoustic energy at a focal point by constructive and destructive interference, as by use of a phased array of acoustic sources to effect constructive interference. A focusing means may be as simple as a solid member having a curved surface, or it may include complex structures such as those found in Fresnel lenses, which employ diffraction in order to direct acoustic radiation.

The term “reservoir” as used herein refers a receptacle or chamber for holding or containing a fluid. Thus, a fluid in a reservoir necessarily has a free surface, i.e., a surface that allows a droplet to be ejected therefrom. As long as a fluid container has at least one free surface from which fluid can be ejected, the container is a reservoir regardless of specific geometry. Thus reservoir contemplates, for example, a microfluidic channel having flowing fluid from which droplets are ejected, and a contained particle plasma. A “cell container” or “cell reservoir” is a reservoir which is specialized for ejection of living cells suspended in a carrier fluid, and includes, by example a microfluidic or other channel through which living cells flow suspended in a carrier fluid.

The term “substrate” as used herein refers to any material having a surface onto which one or more cells contained in a droplet of carrier fluid may be deposited. The substrate may be constructed in any of a number of forms such as wafers, slides, well plates, membranes, for example. In addition, the substrate may be porous or nonporous as may be required for any particular fluid deposition. Suitable substrate materials include, but are not limited to, supports that are typically used for solid phase chemical synthesis, e.g., polymeric materials (e.g., polystyrene, polyvinyl acetate, polyvinyl chloride, polyvinyl pyrrolidone, polyacrylonitrile, polyacrylamide, polymethyl methacrylate, polytetrafluoroethylene, polyethylene, polypropylene, polyvinylidene fluoride, polycarbonate, divinylbenzene styrene-based polymers), agarose (e.g., Sepharose®), dextran (e.g., Sephadex®), cellulosic polymers and other polysaccharides, silica and silica-based materials, glass (particularly controlled pore glass, or “CPG”) and functionalized glasses, ceramics, and such substrates treated with surface coatings, e.g., with microporous polymers (particularly cellulosic polymers such as nitrocellulose and spun synthetic polymers such as spun polyethylene), metallic compounds (particularly microporous aluminum), or the like. While the foregoing support materials are representative of conventionally used substrates, it is to be understood that the substrate may in fact comprise any biological, nonbiological, organic and/or inorganic material, and may be in any of a variety of physical forms, e.g., particles, strands, precipitates, gels, sheets, tubing, spheres, containers, capillaries, pads, slices, films, plates, slides, and the like, and may further have any desired shape, such as a disc, square, sphere, circle, etc. The substrate surface may or may not be flat, e.g., the surface may contain raised or depressed regions.

A substrate may additionally contain or may be derivatized to contain reactive functionality which covalently links a compound to the surface thereof. These are widely known and include, for example, silicon dioxide supports containing reactive Si—OH groups, polyacrylamide supports, polystyrene supports, polyethyleneglycol supports, and the like. Alternatively a moiety which binds to a cognate moiety, for example a ligand receptor pair may be employed to specifically attach a molecule, particle, living cell, biological tissue or tissue component or the like to a substrate surface. One example of attachment using a cognate moiety pair employs a surface that is covalently linked to the ligand biotin, a type of biotin functionalized or biotinylated surface, and the receptor protein streptavidin which specifically binds biotin in a reversible non-covalent manner typical of ligand receptor interactions. Macromolecules such as fusion proteins comprising streptavidin, solid or gel particles to which streptavidin is securely attached and cells transformed to externally display streptavidin may be attached to the biotinylated substrate surface.

The term “surface modification” as used herein refers to the chemical and/or physical alteration of a surface by an additive or subtractive process to change one or more chemical and/or physical properties of a substrate surface or a selected site or region of a substrate surface. For example, surface modification may involve (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface. Thus an example of a surface modification by functionalization is the biotinylated surface that can be used in conjunction with the receptor streptavidin to effect various attachments.

In one embodiment, then, the invention pertains to a device for acoustically ejecting a plurality of single cell containing droplets toward designated sites on a substrate surface. The device comprises a plurality of cell containers or reservoirs, each adapted to contain a carrier fluid within which living cells are suspended; an ejector comprising an acoustic radiation generator for generating acoustic radiation and a focusing means for focusing acoustic radiation at a focal point within and near the fluid surface in each of the reservoirs; and a means for positioning the ejector in acoustic coupling relationship to each of the reservoirs.

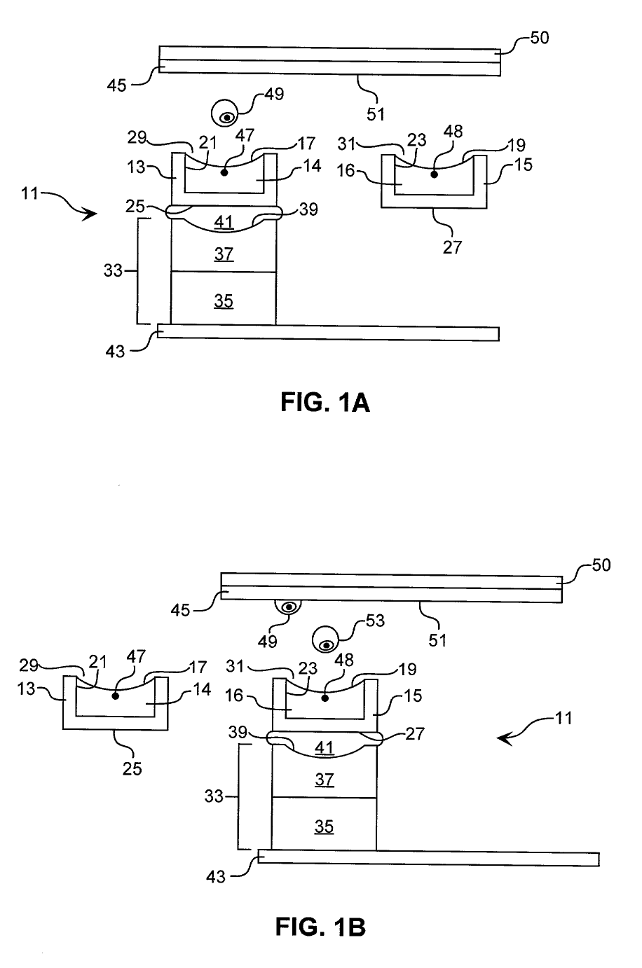

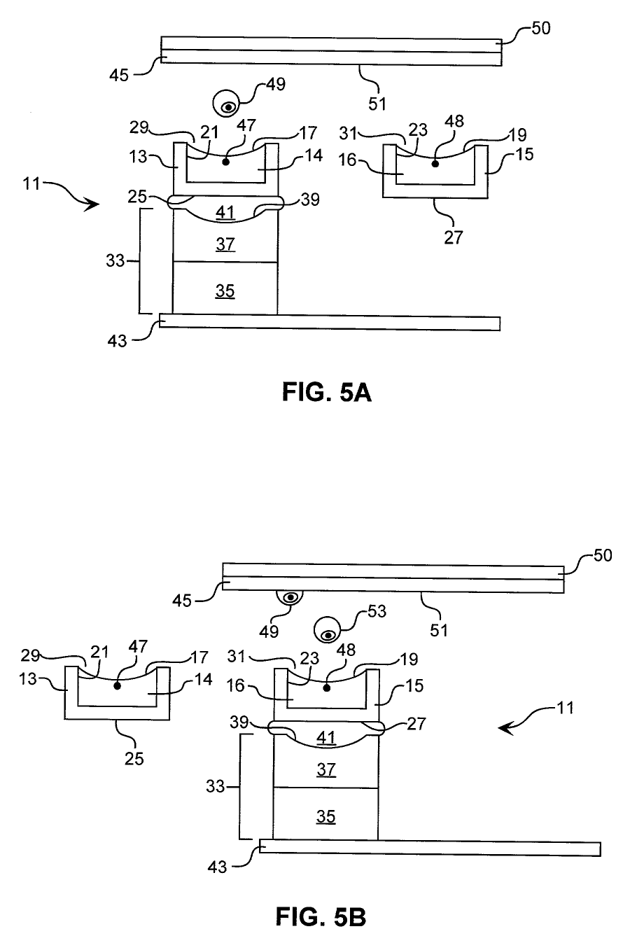

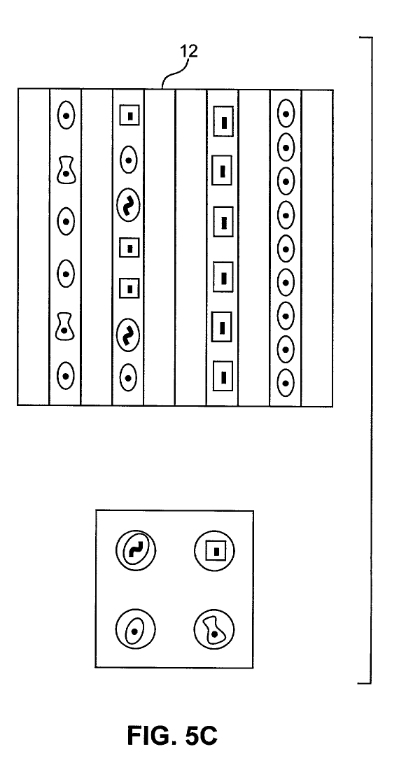

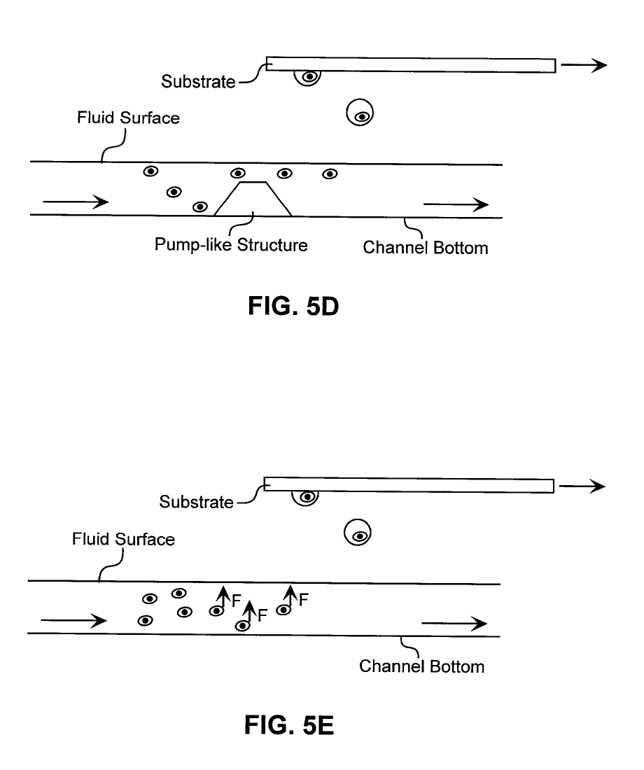

FIGS. 1 and 5 illustrate alternative embodiments of the employed device in simplified cross-sectional view. FIG. 1 depicts a cell ejection system where the cell container or reservoir is a conventional container, such as a conventional petri dish, which is radially symmetric. In FIG. 5, the cell reservoir is a fluidic channel, through which live cells flow in a carrier fluid. As with all figures referenced herein, in which like parts are referenced by like numerals, FIGS. 1 and 5 are not to scale, and certain dimensions may be exaggerated for clarity of presentation. The device 11 includes a plurality of cell containers or reservoirs, i.e., at least two containers or reservoirs, with a first cell container indicated at 13 and a second container indicated at 15, each adapted to contain a fluid, in which live cells are suspended, having a fluid surface, e.g., a first cell container having cells suspended in fluid 14 and a second cell container having cells suspended in fluid 16 having fluid surfaces respectively indicated at 17 and 19. The suspended cells and carrier fluids of 14 and 16 may be the same or different. As depicted, the cell containers or reservoirs are of substantially identical construction so as to be substantially acoustically indistinguishable, but identical construction is not a requirement. The cell containers are shown as separate removable components but may, if desired, be fixed within a plate or other substrate. For example, the plurality of containers in FIG. 1 may comprise individual wells in a well plate, optimally although not necessarily arranged in an array. Likewise, the plurality of containers in FIG. 5 may comprise separate channels or individual channels in a plate, by example a pattern of individual microfluidic channels etched into a plate as by photolithography. Each of the cell containers or reservoirs 13 and 15 is preferably bilaterally (FIG. 5—channels) or axially (FIG. 1) symmetric, having substantially vertical walls 21 and 23 extending upward from reservoir bases 25 and 27 and terminating at openings 29 and 31, respectively, although other reservoir shapes may be used, including enclosed fluidic channels having an aperture or opening for ejection at a specific location. The material and thickness of each cell container or reservoir base should be such that acoustic radiation may be transmitted therethrough and into the fluid contained within the reservoirs.

The device embodiments depicted in FIGS. 1 and 5 also include an acoustic ejector 33 comprised of an acoustic radiation generator 35 for generating acoustic radiation and a focusing means 37 for focusing the acoustic radiation at a focal point within the fluid from which a droplet is to be ejected, near the fluid surface. As shown in FIGS. 1 and 5, the focusing means 37 may comprise a single solid piece having a concave surface 39 for focusing acoustic radiation, but the focusing means may be constructed in other ways as discussed below. The acoustic ejector 33 is thus adapted to generate and focus acoustic radiation so as to eject a droplet of fluid from each of the fluid surfaces 17 and 19 when acoustically coupled to reservoirs 13 and 15 and thus to fluids 14 and 16, respectively. The acoustic radiation generator 35 and the focusing means 37 may function as a single unit controlled by a single controller, or they may be independently controlled, depending on the desired performance of the device. Typically, single ejector designs are preferred over multiple ejector designs because accuracy of droplet placement and consistency in droplet size and velocity are more easily achieved with a single ejector.

As will be appreciated by those skilled in the art, any of a variety of focusing means may be employed in conjunction with the present invention. For example, one or more curved surfaces may be used to direct acoustic radiation to a focal point near a fluid surface. One such technique is described in U.S. Pat. No. 4,308,547 to Lovelady et al. Focusing means with a curved surface have been incorporated into commercially available acoustic transducers such as those manufactured by Panametrics Inc. (Waltham, Mass.). In addition, Fresnel lenses are known in the art for directing acoustic energy at a predetermined focal distance from an object plane. See, e.g., U.S. Pat. No. 5,041,849 to Quate et al. Fresnel lenses may have a radial phase profile that diffracts a substantial portion of acoustic energy into a predetermined diffraction order at diffraction angles that vary radially with respect to the lens. The diffraction angles should be selected to focus the acoustic energy within the diffraction order on a desired object plane. Phased arrays of acoustic energy emitters have also been used to focus acoustic energy at a specified point as a result of constructive and destructive interference between the acoustic waves emitted by the arrayed sources (Amemiya et al (1997) Proceeding of 1997 IS&T NIP13 International Conference on Digital Printing Technologies Proceedings, pp. 698-702.).

There are also a number of ways to acoustically couple the ejector 33 to each individual reservoir and thus to the fluid therein. One such approach is through direct contact as is described, for example, in U.S. Pat. No. 4,308,547 to Lovelady et al., wherein a focusing means constructed from a hemispherical crystal having segmented electrodes is submerged in a liquid to be ejected. The aforementioned patent further discloses that the focusing means may be positioned at or below the surface of the liquid. However, this approach for acoustically coupling the focusing means to a fluid is undesirable when the ejector is used to eject different fluids in a plurality of containers or reservoirs, as repeated cleaning of the focusing means would be required in order to avoid cross-contamination. The cleaning process would necessarily lengthen the transition time between each droplet ejection event. In addition, in such a method, cells in the fluid would adhere to the ejector as it is removed from a container, wasting cellular material that may be rare or irreplaceable. Finally, submersion in the fluid is not possible with conventional acoustic energy focusing means when the reservoirs are microfabricated, as when the cell containers are microfluidic channels or micro-wells, because of size difference, the containers being too small.

One of skill in the art of microfabrication would be able to make a focusing means comprising a microfabricated curved member. Similarly a microfabricated focusing means constructed from a hemispherical crystal having segmented electrodes, e.g. a miniature focusing means as described in U.S. Pat. No. 4,308,547 to Lovelady et al., can be made by routine microfabrication techniques. Submersion would then be possible with the same disadvantages as above. For microfluidic channels or wells, then, a focusing means as well as a source of acoustic energy could be integrated into the microfabricated assembly.

An approach practicable for any reservoir dimensions would be to acoustically couple a conventional non-microfabricated or macro-scale ejector to the reservoirs and reservoir fluids without contacting any portion of the ejector, e.g., the focusing means, with any of the fluids to be ejected. To this end, the present invention provides an ejector positioning means for positioning the ejector in controlled and repeatable acoustic coupling with each of the fluids in the cell containers or reservoirs to eject droplets therefrom without submerging the ejector therein. This typically involves direct or indirect contact between the ejector and the external surface of each reservoir. When direct contact is used in order to acoustically couple the ejector to each reservoir, it is preferred that the direct contact is wholly conformal to ensure efficient acoustic energy transfer. That is, the ejector and the reservoir should have corresponding surfaces adapted for mating contact. Thus, if acoustic coupling is achieved between the ejector and reservoir through the focusing means, it is desirable for the reservoir to have an outside surface that corresponds to the surface profile of the focusing means. Without conformal contact, efficiency and accuracy of acoustic energy transfer may be compromised. In addition, since many focusing means have a curved surface, the direct contact approach may necessitate the use of reservoirs having a specially formed inverse surface.

Optimally, acoustic coupling is achieved between the ejector and each of the reservoirs through indirect contact, as illustrated in FIGS. 1A and 5A. In the figure, an acoustic coupling medium 41 is placed between the ejector 33 and the base 25 of reservoir 13, with the ejector and reservoir located at a predetermined distance from each other. The acoustic coupling medium may be an acoustic coupling fluid, preferably an acoustically homogeneous material in conformal contact with both the acoustic focusing means 37 and each reservoir. In addition, it is important to ensure that the fluid medium is substantially free of material having different acoustic properties than the fluid medium itself. As shown, the first reservoir 13 is acoustically coupled to the acoustic focusing means 37 such that an acoustic wave is generated by the acoustic radiation generator and directed by the focusing means 37 into the acoustic coupling medium 41, which then transmits the acoustic radiation into the reservoir 13.

In operation, reservoirs 13 and 15 of the device are each filled with first and second carrier fluids having cells or cell mixtures suspended therein 14 and 16, respectively, as shown in FIGS. 1 and 5. The acoustic ejector 33 is positionable by means of ejector positioning means 43, shown below reservoir 13, in order to achieve acoustic coupling between the ejector and the reservoir through acoustic coupling medium 41. Substrate 45 is positioned above and in proximity to the first reservoir 13 such that one surface of the substrate, shown in FIGS. 1 and 5 as underside surface 51, faces the reservoir and is substantially parallel to the surface 17 of the fluid 14 therein. Once the ejector, the reservoir and the substrate are in proper alignment, the acoustic radiation generator 35 is activated to produce acoustic radiation that is directed by the focusing means 37 to a focal point 47 near the fluid surface 17 of the first reservoir. As a result, droplet 49 is ejected from the fluid surface 17 onto a designated site on the underside surface 51 of the substrate. The ejected droplet may be retained on the substrate surface by solidifying thereon after contact; in such an embodiment, it is necessary to maintain the substrate at a low temperature, i.e., a temperature that results in droplet solidification after contact. Alternatively, or in addition, a molecular moiety within the droplet attaches to the substrate surface after contract, through adsorption, physical immobilization, or covalent binding.

Then, as shown in FIGS. 1B and 5B, a substrate positioning means 50 repositions the substrate 45 over reservoir 15 in order to receive a droplet therefrom at a second designated site. FIGS. 1B and 5B also show that the ejector 33 has been repositioned by the ejector positioning means 43 below reservoir 15 and in acoustically coupled relationship thereto by virtue of acoustic coupling medium 41. Once properly aligned as shown in FIGS. 1B and 5B, the acoustic radiation generator 35 of ejector 33 is activated to produce acoustic radiation that is then directed by focusing means 37 to a focal point within fluid 16 near the fluid surface 19, thereby ejecting droplet 53 onto the substrate. It should be evident that such operation is illustrative of how the employed device may be used to eject a plurality of single cells contained in fluid droplets from reservoirs in order to form a pattern, e.g., an array, of cells on the substrate surface 51. It should be similarly evident that the device may be adapted to eject a plurality of individual cells contained in ejected fluid droplets from one or more reservoirs onto the same site of the substrate surface.

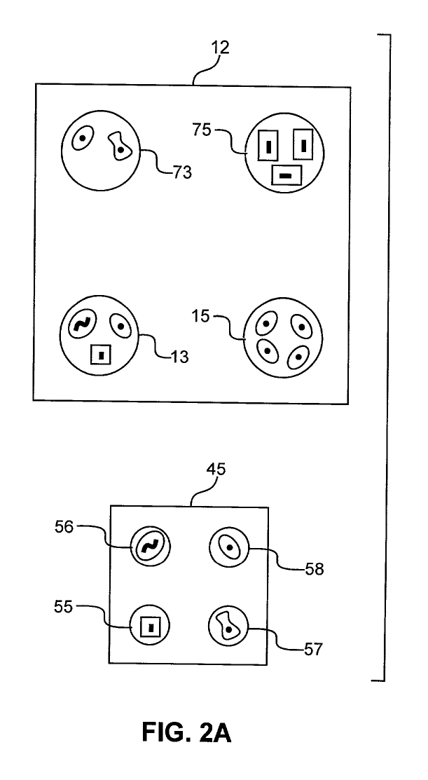

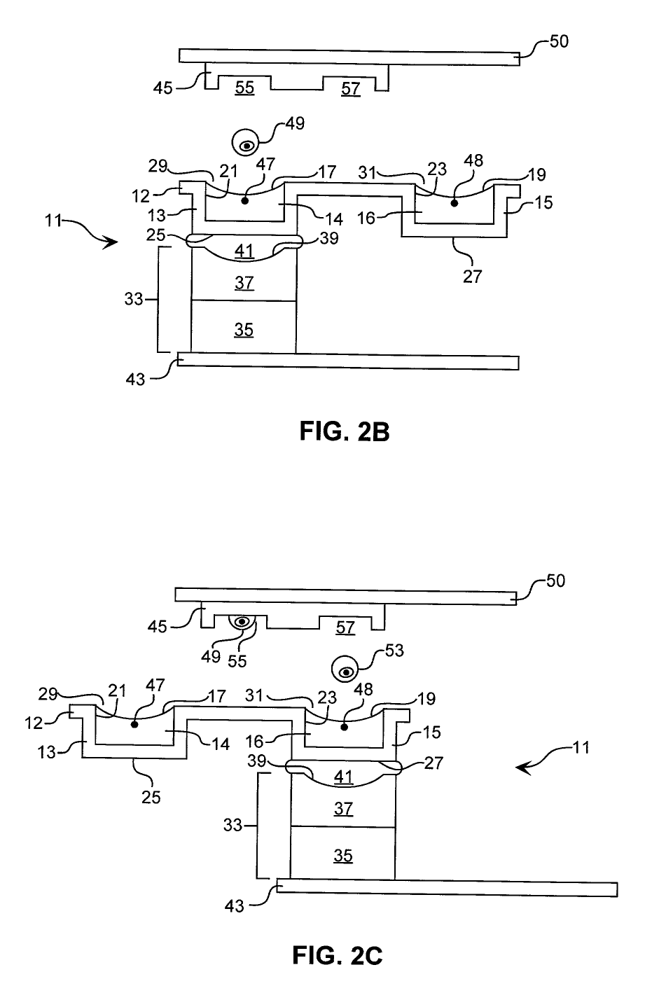

In another embodiment, the device is constructed so as to allow transfer of cells contained in fluid droplets between well plates, in which case the substrate comprises a substrate well plate, and the fluid suspended cell-containing reservoirs are individual wells in a reservoir well plate. FIG. 2 illustrates such a device, wherein four individual wells 13, 15, 73 and 75 in reservoir well plate 12 serve as fluid reservoirs for containing a plurality of a specific type of cell or a mixture of different cell types suspended in a fluid for ejection of droplets containing a single cell, and the substrate comprises a smaller well plate 45 of four individual wells indicated at 55, 56, 57 and 58. FIG. 2A illustrates the cell container or reservoir well plate and the substrate well plate in top plane view. As shown, each of the well plates contains four wells arranged in a two-by-two array. FIG. 2B illustrates the employed device wherein the cell container or reservoir well plate and the substrate well plate are shown in cross-sectional view along wells 13, 15 and 55, 57, respectively. As in FIGS. 1 and 5, reservoir wells 13 and 15 respectively contain cells suspended in carrier fluids 14 and 16 having carrier fluid surfaces respectively indicated at 17 and 19. The materials and design of the wells of the cell container or reservoir well plate are similar to those of the containers illustrated in FIGS. 1 and 5. For example, the cell containers or reservoirs shown in FIG. 2B (wells) and in FIG. 5B (channels) are of substantially identical construction so as to be substantially acoustically indistinguishable. In these embodiments, the bases of the cell reservoirs are of a material (e.g. a material having appropriate acoustic impedance) and thickness so as to allow efficient transmission of acoustic radiation therethrough into the contained carrier fluid.

The device of FIGS. 2 and 5 also includes an acoustic ejector 33 having a construction similar to that of the ejector illustrated in FIG. 1, comprising an acoustic generating means 35 and a focusing means 37. FIG. 2B shows the ejector acoustically coupled to a reservoir well through indirect contact; that is, an acoustic coupling medium 41 is placed between the ejector 33 and the reservoir well plate 12, i.e., between the curved surface 39 of the acoustic focusing means 37 and the base 25 of the first cell container or reservoir (well or channel) 13. As shown, the first cell container or reservoir (well or channel) 13 is acoustically coupled to the acoustic focusing means 37 such that acoustic radiation generated in a generally-upward direction is directed by the focusing means 37 into the acoustic coupling medium 41, which then transmits the acoustic radiation into the cell container or reservoir (well or channel) 13.

In operation, each of the cell containers or reservoirs (well or channel) is preferably filled with a carrier fluid having a different type of cell or mixture of cells suspended within the carrier fluid. As shown, reservoir wells 13 and 15 of the device are each filled with a carrier fluid having a first cell mixture 14 and a carrier fluid having a second cell mixture 16, as in FIG. 1, to form fluid surfaces 17 and 19, respectively. FIGS. 1 and 5 show that the ejector 33 is positioned below reservoir well 13 by an ejector positioning means 43 in order to achieve acoustic coupling therewith through acoustic coupling medium 41.

For the ejection of individual cells into well plates from cell containers, FIG. 2A, the first substrate well 55 of substrate well plate 45 is positioned above the first reservoir well 13 in order to receive a droplet ejected from the first cell container or reservoir (well or channel).

Once the ejector, the cell container or reservoir (well or channel) and the substrate are in proper alignment, the acoustic radiation generator is activated to produce an acoustic wave that is focused by the focusing means to direct the acoustic wave to a focal point 47 near fluid surface 17, with the amount of energy being insufficient to eject fluid. This first emission of focused acoustic energy permits sonic detection of the presence of a cell sufficiently close to the surface for ejection by virtue of reflection of acoustic energy created by a difference in acoustic impedance between the cell and carrier fluid. After a cell is detected and localized other properties may be measured before the decision to eject is made. Also, if no cell is sufficiently close to the surface for ejection, the acoustic energy may be focused at progressively greater distances from the fluid surface until a cell is located and driven closer to the surface by focused acoustic energy or other means such as a photon field. Alternatively, a uniform field such as a photon field which will exert a force based on cross sectional area and change in photon momentum, determined by the difference of refractive indices of the carrier medium and the cells, or an electric field, exerting a force based on net surface charge, a carrier fluid having a low density relative to the cells or a carrier fluid comprising a density gradient. It will be appreciated that numerous ways of effecting a short mean cell distance from the fluid surface exists. For channels, especially microfabricated channels, mechanical means may be used to effect a sufficiently small distance from the fluid surface by placing a ramp like structure across the channel that decreases channel depth over the ramp to a depth on the order of the cell diameter, thereby only permitting cells to flow near the surface; cells are unlikely to jam at the ramp because the fluid velocity will be highest where the channel depth is lowest as depicted in FIG. 5D. FIG. 5E depicts a microfluidic channel where a force acting on the cells moves them towards the surface.

Because microfluidic channels may be fabricated with small dimensions that reduce the volume in which a cell may be located, they are especially preferred for use with acoustic ejection as locating a cell suitable for ejection is greatly simplified. For example, for a cell type or mixture of cell types having relatively uniform size, for example mean diameter of 10.0 μm, SD≈0.5 μm, the channel can be engineered to be about 12.0 μm wide and deep, effecting a single file of cells uniformly a mean distance of about 1.0 μm from the fluid surface (ejection volume≈4/3πr³=0.52 pL), without for example providing a ramp (FIG 5D) or otherwise promoting a short distance between surface and cell location as by the preceding methods that effect a net upwards force on the cells. The cells can be ejected from the channel at a certain limited distance range along the fluid flow axis, reducing the area of fluid surface scanned. For example a 50 μm aperture for ejecting cells can be provided in a closed capillary, or a limited distance along the flow axis of an open capillary may be used for ejection, a significant advantage being that the cells move past the ejector, reducing the area scanned for cells. Even when employing such methods to float cells in a macro-scale container such as a petri dish, significant amounts of time will be wasted scanning in the plane parallel to the fluid surface to locate a cell to eject. The advantages of employing microfluidic channels are only slightly diminished for a wider range of cell sizes for example, red blood cells (RBC, mean diameter of 7 μm, SD≈0.3 μm, biconcave disc, height≈3 μm) mixed with the preceding cell type (mean diameter of 10.0 μm, SD≈0.5 μm). Although the RBCs can be a significant depth from the surface relative to the fluid ejection volume and corresponding energy required to eject a RBC, this can be overcome by the described methods of forcing cells toward the fluid surface, and the advantage of limiting the lateral search to about 12 μm width as opposed to several cm wide petri dish is immediately apparent

Once a cell sufficiently close to the surface is located and determined to meet any other criteria for ejection, the acoustic radiation generator is activated to produce an acoustic wave that is focused by the focusing means to direct the acoustic wave to a focal point 47 near fluid surface 17, with the amount of energy being sufficient to eject a volume of fluid substantially corresponding to the volume of the cell or cells to be ejected so that any ejected volume does not contain more than one cell. The precise amount of energy required to eject only the required volume and no more can be initially calibrated by slowly increasing the energy applied from an amount insufficient to eject a cell desired for ejection until there is just enough energy applied to eject the cell the desired distance to the targeted substrate locale. After this initial calibration approximately the same energy, with adjustment for any change in fluid level, may be applied to eject cells of substantially the same volume as the initial calibration cell. As a result, droplet 49, containing a single living cell, is ejected from fluid surface 17 into the first substrate well 55 of the substrate well plate 45. The cell containing droplet is retained on the substrate well plate by surface tension.

Then, as shown in FIG. 2C, the substrate well plate 45 is repositioned by a substrate positioning means 50 such that substrate well 57 is located directly over cell container or reservoir (well or channel) 15 in order to receive a cell containing droplet therefrom. FIG. 2C also shows that the ejector 33 has been repositioned by the ejector positioning means below cell container well 15 to acoustically couple the ejector and the container through acoustic coupling medium 41. Since the substrate well plate and the reservoir well plate or channels on a planar substrate are differently sized, there is only correspondence, not identity, between the movement of the ejector positioning means and the movement of the substrate well plate. Once properly aligned as shown in FIG. 2C, the acoustic radiation generator 35 of ejector 33 is activated to produce an acoustic wave that is then directed by focusing means 37 to a focal point near the fluid surface 19 for detection of the presence of a cell sufficiently close to the carrier fluid surface for ejection. After detection and measurement of any property forming a criterion for ejection, the acoustic radiation generator 35 of ejector 33 is activated to produce an acoustic wave that is then directed by focusing means 37 to a focal point near the fluid surface 19 from which cell containing droplet 53 is ejected onto the second well of the substrate well plate. It should be evident that such operation is illustrative of how the employed device may be used to transfer a plurality of single cells contained in appropriately sized droplets from one well plate to another of a different size. One of ordinary skill in the art will recognize that this type of transfer may be carried out even when the cells, the carrier fluid and both the ejector and substrate are in continuous motion. It should be further evident that a variety of combinations of reservoirs, well plates and/or substrates may be used in using the employed device to engage in single cell containing fluid droplet transfer. It should be still further evident that any reservoir may be filled with a fluid carrier or cells suspended in a fluid carrier through acoustic ejection of cell containing or cell free fluid droplets respectively prior to deploying the reservoir for further transfer of fluid droplets containing cells, e.g., for cell array deposition.

As discussed above, either individual, e.g., removable, reservoirs (well or channel) or plates (well or channel) may be used to contain cell suspensions in carrier fluids that are to be ejected, wherein the reservoirs or the wells of the well plate are preferably substantially acoustically indistinguishable from one another. Also, unless it is intended that the ejector is to be submerged in the fluid to be ejected, the reservoirs or well plates must have acoustic transmission properties sufficient to allow acoustic radiation from the ejector to be conveyed to the surfaces of the fluids to be ejected. Typically, this involves providing reservoir or well bases that are sufficiently thin relative to the acoustic impedance of the material from which they are made, to allow acoustic radiation to travel therethrough without unacceptable dissipation. In addition, the material used in the construction of reservoirs must be compatible with the contained carrier fluids, and non-toxic to the suspended cells.

Thus, as it is intended that the reservoirs or wells contain live cells suspended in an aqueous carrier fluid materials that dissolve or swell in water or release compounds toxic to living cells into the aqueous carrier would be unsuitable for use in forming the reservoirs or well plates. For water-based fluids, a number of materials are suitable for the construction of reservoirs and include, but are not limited to, ceramics such as silicon oxide and aluminum oxide, metals such as stainless steel and platinum, and polymers such as polyester and polytetrafluoroethylene; these materials may be prepared so that substances toxic to cells do not leach into the carrier fluid sufficient amounts to render the carrier fluid toxic to the cells. Many well plates suitable for use with the employed device are commercially available and may contain, for example, 96, 384 or 1536 wells per well plate. Manufactures of suitable well plates for use in the employed device include Coming Inc. (Corning, N.Y.) and Greiner America, Inc. (Lake Mary, Fla.). However, the availability such commercially available well plates does not preclude manufacture and use of custom-made well plates containing at least about 10,000 wells, or as many as 100,000 wells or more. For array forming applications, it is expected that about 100,000 to about 4,000,000 reservoirs may be employed. In addition, to reduce the amount of movement needed to align the ejector with each reservoir or reservoir well, it is preferable that the center of each reservoir is located not more than about 1 centimeter, preferably not more than about 1 millimeter and optimally not more than about 0.5 millimeter from any other reservoir center.

Generally, the device may be adapted to eject fluids of virtually any type and amount desired. Ejected fluid may be aqueous and/or nonaqueous, but only aqueous fluids are compatible with transfer of living cells. Examples aqueous fluids including water per se and water solvated ionic and non-ionic solutions and suspensions or slurries of solids, gels or discrete cells in aqueous liquids. Because of the precision that is possible using the inventive technology, the device may be used to eject droplets from a reservoir adapted to contain no more than about 100 nanoliters of fluid, preferably no more than 10 nanoliters of fluid. In certain cases, the ejector may be adapted to eject a droplet from a reservoir adapted to contain about 1 to about 100 nanoliters of fluid. This is particularly useful when the fluid to be ejected contains rare or expensive biomolecules or cells, wherein it may be desirable to eject droplets having a volume of about up to 1 picoliter.

From the above, it is evident that various components of the device may require individual control or synchronization to form an array of cells on a substrate. For example, the ejector positioning means may be adapted to eject droplets from each cell container or reservoir in a predetermined sequence associated with an array to be prepared on a substrate surface. Similarly, the substrate positioning means for positioning the substrate surface with respect to the ejector may be adapted to position the substrate surface to receive droplets in a pattern or array thereon. Either or both positioning means, i.e., the ejector positioning means and the substrate positioning means, may be constructed from, e.g., levers, pulleys, gears, linear motors a combination thereof, or other mechanical means known to one of ordinary skill in the art. It is preferable to ensure that there is a correspondence between the movement of the substrate, the movement of the ejector and the activation of the ejector to ensure proper pattern formation.

Moreover, the device may include other components that enhance performance. For example, as alluded to above, the device may further comprise cooling means for lowering the temperature of the substrate surface to ensure, for example, that the ejected droplets adhere to the substrate, and rapidly freeze the cells to maintain their viability. The cooling means may be adapted to maintain the substrate surface at a temperature that allows fluid to partially or preferably completely freeze shortly after the cell containing fluid droplet comes into contact therewith. In the case of aqueous fluid droplets containing cells, the cooling means should have the capacity to maintain the substrate surface at no more than about 0° C., preferably much colder. In addition, repeated application of acoustic energy to a reservoir of fluid may result in heating of the fluid. Heating can of course result in unwanted effects on living cells. Thus, the device may further comprise means for maintaining fluid in the cell containers or reservoirs at a constant temperature. Design and construction of such temperature maintaining means are known to one of ordinary skill in the art and may comprise, e.g., components such a heating element, a cooling element, or a combination thereof. For biomolecular and live cell deposition applications, it is generally desired that the fluid containing the biomolecule or cells is kept at a constant temperature without deviating more than about 1° C. or 2° C. therefrom. In addition, for live cells, it is preferred that the fluid be kept at a temperature that does not exceed about 1° C. above the normal temperature from which the cell is derived in the case of warm blooded organisms, and at about 16° C.±about 1° C. for all other organisms whether prokaryotic or eukaryotic, except, for all organisms, in the case that the specific cell type is known to have poor viability unless chilled. Cells that require chilling for viability will be appreciated by those of ordinary skill in the art of culturing and maintaining cells to require a saline carrier fluid of appropriate osmolality (slightly hyperosmotic) at about −1° C.±. Thus, for example, when the biomolecule-containing fluid is aqueous, it may be optimal to keep the fluid at about 4° C. during ejection.

The invention may involve modification of a substrate surface prior to acoustic ejection of cell containing fluid droplets thereon. Surface modification may involve functionalization or defunctionalization, smoothing or roughening, coating, degradation, passivation or otherwise altering the surface's chemical composition or physical properties. In one embodiment the invention requires functionalization with a cognate moiety to an externally displayed marker moiety, but other surface modifications described may affect the success of the inventive method in a specific context.

One such surface modification method involves altering the wetting properties of the surface, for example to facilitate confinement of a cell contained in a droplet ejected onto the surface within a designated area or enhancement of the kinetics for the surface attachment of molecular moieties for functionalizing the substrate or a specific substrate locale, as by patterning biotinylation by acoustic ejection of a biotinylating solution. A preferred method for altering the wetting properties of the substrate surface involves deposition of droplets of a suitable surface modification fluid at each designated site of the substrate surface prior to acoustic ejection of fluids to form an array thereon. In this way, the “spread” of the acoustically ejected droplets and contained cells may be optimized and consistency in spot size (i.e., diameter, height and overall shape) ensured. One way to implement the method involves acoustically coupling the ejector to a modifier reservoir containing a surface modification fluid and then activating the ejector, as described in detail above, to produce and eject a droplet of surface modification fluid toward a designated site on the substrate surface. The method is repeated as desired to deposit surface modification fluid at additional designated sites. Similarly by the methods of copending applications (“Focused Acoustic Energy in the Preparation of Combinatorial Composition of Matter Libraries” U.S. Ser. No. ______, inventors Mutz and Ellson, filed on even date herewith, and “Focused Acoustic Energy in the Preparation of Peptidic Arrays,” U.S. Ser. No. 09/669,997, inventors Mutz and Ellson, filed on Sep. 25, 2000, both of which are assigned to Picoliter, Inc. (Cupertino, Calif.)) or by other methods of generating arrays of biomolecules attached or linked to a substrate surface, cognate moieties that specifically bind to marker moieties displayed on the surface of transformed or untransforned cells may be patterned on the substrate surface. Alternatively a single cognate moiety such as biotin can be linked to the substrate surface either uniformly, or in a pattern, such as biotinylated areas surrounded by non-biotinylated areas, and the cells to be patterned can be transformed to display streptavidin on their surface.

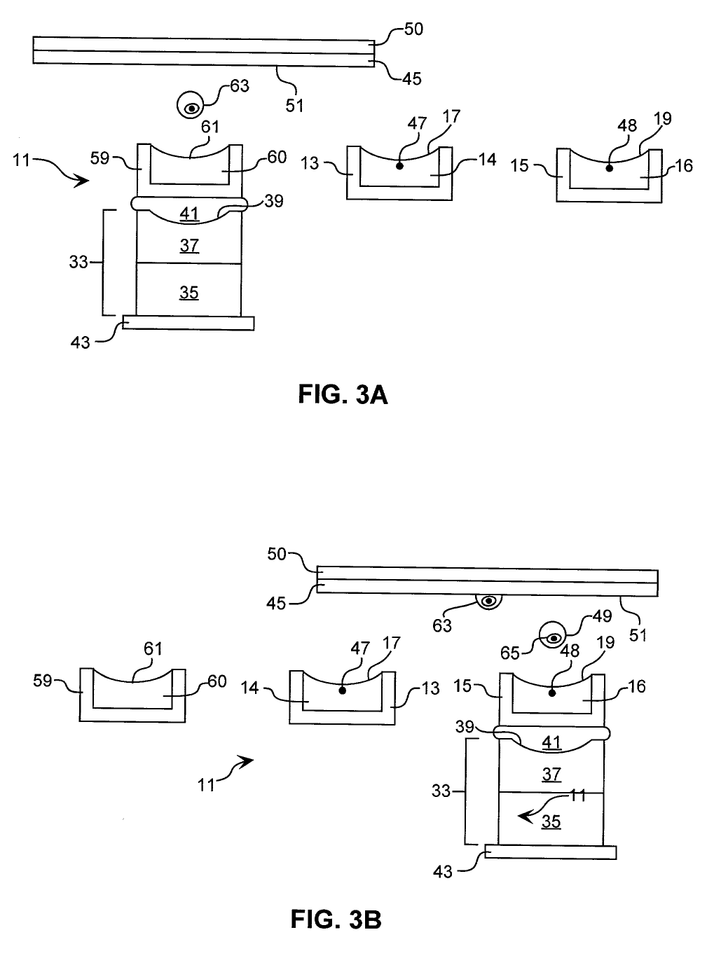

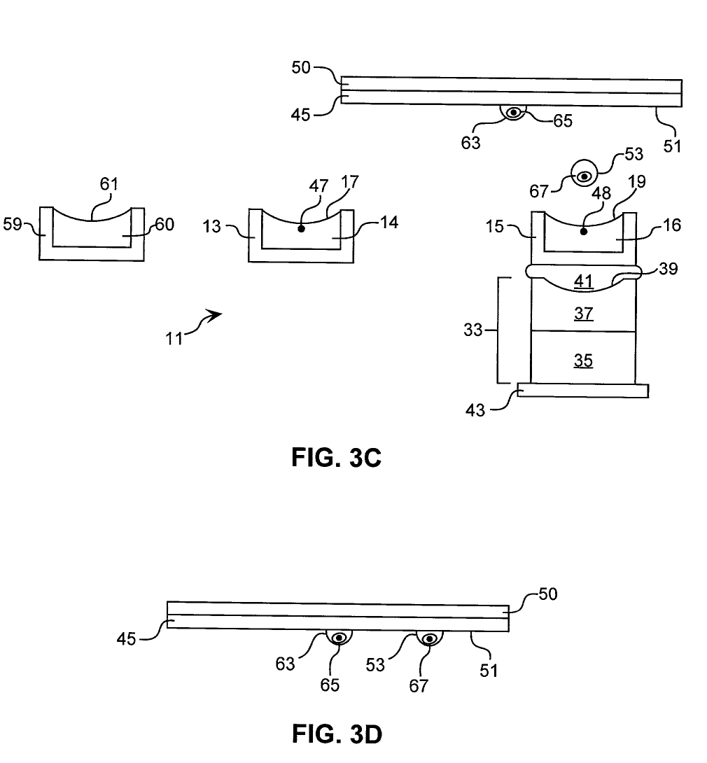

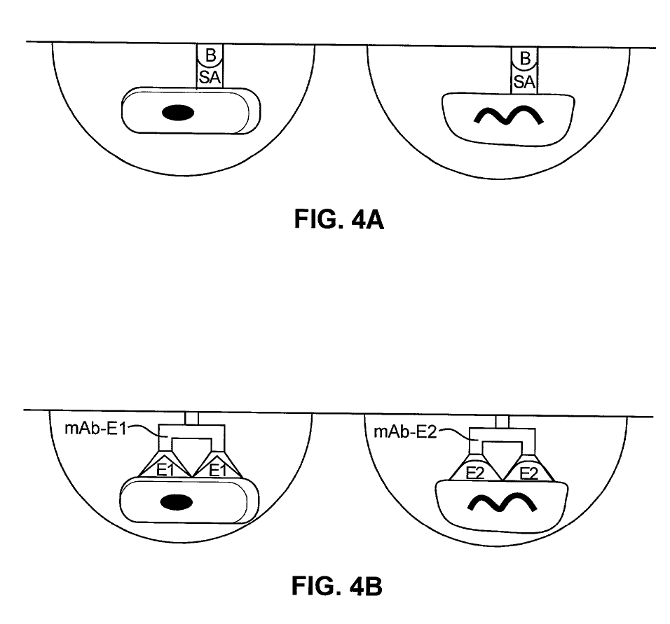

FIG. 3 schematically illustrates in simplified cross-sectional view a specific embodiment of the aforementioned method in which a dimer is synthesized on a substrate using a device similar to that illustrated in FIG. 1, but including a modifier reservoir 59 containing a surface modification fluid 60 having a fluid surface 61. FIG. 3A illustrates the ejection of a droplet 63 of surface modification fluid 60 selected to alter the wetting properties of a designated site on surface 51 of the substrate 45 where the dimer is to be synthesized. The ejector 33 is positioned by the ejector positioning means 43 below modifier reservoir 59 in order to achieve acoustic coupling therewith through acoustic coupling medium 41. Substrate 45 is positioned above the modifier reservoir 19 at a location that enables acoustic deposition of a droplet of surface modification fluid 60 at a designated site. Once the ejector 33, the modifier reservoir 59 and the substrate 45 are in proper alignment, the acoustic radiation generator 35 is activated to produce acoustic radiation that is directed by the focusing means 37 in a manner that enables ejection of droplet 63 of the surface modification fluid 60 from the fluid surface 61 onto a designated site on the underside surface 51 of the substrate. Once the droplet 63 contacts the substrate surface 51, the droplet modifies an area of the substrate surface to result in an increase or decrease in the surface energy of the area with respect to deposited fluids.

Then, as shown in FIG. 3B, the substrate 45 is repositioned by the substrate positioning means 50 such that the region of the substrate surface modified by droplet 63 is located directly over reservoir 13. FIG. 3B also shows that the ejector 33 is positioned by the ejector positioning means below reservoir 13 to acoustically couple the ejector and the reservoir through acoustic coupling medium 41. Once properly aligned, the ejector 33 is again activated so as to eject droplet 49 onto substrate. Droplet 49 contains a single cell 65, preferably displaying a marker moiety on its external cell membrane that is specifically bound by a cognate moiety linked to the surface to effect specific attachment to the surface. The marker moiety may occur in an untransformed cell or may be the result of transformation or genetic manipulation, and may optionally signify transformation to express a gene other than the marker, e.g. as a reporter of transformation with another gene.

Then, as shown in FIG. 3C, the substrate 45 is again repositioned by the substrate positioning means 50 such that a different site than the site having the first single cell 65 attached thereto is located directly over reservoir 15 in order to receive a cell contained in a droplet therefrom. FIG. 3B also shows that the ejector 33 is positioned by the ejector positioning means below reservoir 15 to acoustically couple the ejector and the reservoir through acoustic coupling medium 41. Once properly aligned, the ejector 33 is again activated so that droplet 53 is ejected onto substrate. Droplet 53 contains a second single cell.

Often cognate moieties are ligands including oligonucleotides and peptides. Marker moieties are likely to be peptides or peptidoglycans. The chemistry employed in synthesizing substrate-bound oligonucleotides can be adapted to acoustic fluid droplet ejection (see co-pending patent application U.S. Ser. No. 09/669,996, entitled “Acoustic Ejection of Fluids from a Plurality of Reservoirs,” inventors Mutz and Ellson, filed on Aug. 25, 2000 and assigned to Picoliter, Inc. (Cupertino, Calif.)). These methods may be used to create arrays of oligonucleotides on a substrate surface for use with the instant invention. Such adaptation will generally involve now-conventional techniques known to those skilled in the art of nucleic acid chemistry and/or described in the pertinent literature and texts. See, for example, DNA Microarrays: A Practical Approach, M. Schena, Ed. (Oxford University Press, 1999). That is, the individual coupling reactions are conducted under standard conditions used for the synthesis of oligonucleotides and conventionally employed with automated oligonucleotide synthesizers. Such methodology is described, for example, in D.M. Matteuci et al. (1980) Tet. Lett. 521:719, U.S. Pat. No. 4,500,707 to Caruthers et al., and U.S. Pat. Nos. 5,436,327 and 5,700,637 to Southern et al. Focused acoustic energy may also be adapted to in situ combinatorial oligonucleotide, oligopeptide and oligosaccharide syntheses for forming combinatorial arrays for use with the instant invention (see co-pending patent application U.S. Ser. No. ______, entitled “Focused Acoustic Energy in the Preparation and Screening of Combinatorial Composition of Matter Libraries,” inventors Mutz and Ellson, referenced supra).

Alternatively, an oligomer may be synthesized prior to attachment to the substrate surface and then “spotted” onto a particular locus on the surface using the methodology of the invention. Again, the oligomer may be an oligonucleotide, an oligopeptide, oligosaccharide or any other biomolecular (or nonbiomolecular) oligomer moiety. Preparation of substrate-bound peptidic molecules, e.g., in the formation of peptide arrays and protein arrays, is described in copending patent application U.S. Ser. No. 09/669,997 (“Focused Acoustic Energy in the Preparation of Peptidic Arrays”), inventors Mutz and Ellson, filed on Sep. 25, 2000 and assigned to Picoliter, Inc. (Cupertino, Calif.). Preparation of substrate-bound oligonucleotides, particularly arrays of oligonucleotides wherein at least one of the oligonucleotides contains partially nonhybridizing segments, is described in co-pending patent application U.S. Ser. No. 09/669,267 (“Arrays of Oligonucleotides Containing Nonhybridizing Segments”), inventor Ellson, also filed on Sep. 25, 2000 and assigned to Picoliter, Inc.

These acoustic ejection methods enable preparation of molecular arrays, particularly biomolecular arrays, having densities substantially higher than possible using current array preparation techniques such as photolithographic processes, piezoelectric techniques (e.g., using inkjet printing technology), and microspotting, for use with the instant invention. The array densities that may be achieved using the devices and methods of the invention are at least about 1,000,000 biomolecules per square centimeter of substrate surface, preferably at least about 1,500,000 per square centimeter of substrate surface. The biomolecular moieties may be, e.g., peptidic molecules and/or oligonucleotides. Often such densities are not necessary for creating sites containing individual cells, which are separated by a distance from other cells. But adaptation of such methods, for example, to functionalize a discrete portion of a site surface with cognate moieties which specifically bind a marker moiety, may be useful in localizing the cells within the site, or for situations where the cells are deliberately arrayed in close proximity. For example, for a lymphocyte array (small≈8 μm, medium≈12 μm, large≈14 μm), when the sites are 100 μm=100 μm squares, functionalizing a 10 μm diameter spot in the center of each site with the appropriate cognate moiety to specifically bind the spotted cell will ensure sufficient cell separation to allow, for example testing or screening of individual cells by acoustic deposition of reagent containing fluid droplets of sufficient volume to expose or treat the cell without necessarily exposing cells at adjacent sites to the same condition, permitting, for example, combinatorial screening of cells.

It should be evident, then, that many variations of the invention are possible. For example, each of the ejected cell containing droplets may be deposited as an isolated and “final” feature. Alternatively, or in addition, a plurality of ejected droplets, each containing one or a plurality of cells may be deposited on the same location of a substrate surface in order to synthesize a cell array where each site contains multiple cells of either known or unknown but ascertainable number, or to pattern cells for other purposes such as tissue engineering on a pattern replicating a specific histologic architecture. For cell array and patterning fabrication employing attachment, it is expected that washing steps may be used between droplet ejection steps. Such wash steps may involve, e.g., submerging the entire substrate surface on which cells have been deposited in a washing fluid.

The invention enables ejection of droplets at a rate of at least about 1,000,000 droplets per minute from the same reservoir, and at a rate of at least about 100,000 drops per minute from different reservoirs. In addition, current positioning technology allows for the ejector positioning means to move from one cell container or reservoir to another quickly and in a controlled manner, thereby allowing fast and controlled ejection of different fluids. That is, current commercially available technology allows the ejector to be moved from one reservoir to another, with repeatable and controlled acoustic coupling at each reservoir, in less than about 0.1 second for high performance positioning means and in less than about 1 second for ordinary positioning means. A custom designed system will allow the ejector to be moved from one reservoir to another with repeatable and controlled acoustic coupling in less than about 0.001 second. In order to provide a custom designed system, it is important to keep in mind that there are two basic kinds of motion: pulse and continuous. Pulse motion involves the discrete steps of moving an ejector into position, emitting acoustic energy, and moving the ejector to the next position; again, using a high performance positioning means with such a method allows repeatable and controlled acoustic coupling at each reservoir in less than 0.1 second. A continuous motion design, on the other hand, moves the ejector and the reservoirs continuously, although not at the same speed, and provides for ejection during movement. Since the pulse width is very short, this type of process enables over 10 Hz reservoir transitions, and even over 1000 Hz reservoir transitions.

In order to ensure the accuracy of fluid ejection, it is important to determine the location and the orientation of the fluid surface from which a droplet is to be ejected with respect to the ejector. Otherwise, ejected droplets may be improperly sized or travel in an improper trajectory. Thus, another embodiment of the invention relates to a method for determining the height of a fluid surface and the proximity of a cell in a reservoir between ejection events. The method involves acoustically coupling a fluid-containing reservoir to an acoustic radiation generator and activating the generator to produce a detection acoustic wave that travels to the fluid surface and is reflected thereby as a reflected acoustic wave. Parameters of the reflected acoustic radiation are then analyzed in order to assess the spatial relationship between the acoustic radiation generator and the fluid surface. Such an analysis will involve the determination of the distance between the acoustic radiation generator and the fluid surface and/or the orientation of the fluid surface in relationship to the acoustic radiation generator.

More particularly, the acoustic radiation generator may activated so as to generate low energy acoustic radiation that is insufficiently energetic to eject a droplet from the fluid surface. This is typically done by using an extremely short pulse (on the order of tens of nanoseconds) relative to that normally required for droplet ejection (on the order of microseconds). By determining the time it takes for the acoustic radiation to be reflected by the fluid surface back to the acoustic radiation generator and then correlating that time with the speed of sound in the fluid, the distance—and thus the fluid height—may be calculated; the presence distance of a cell beneath the surface can be determined likewise. Of course, care must be taken in order to ensure that acoustic radiation reflected by the interface between the reservoir base and the fluid is discounted. It will be appreciated by those of ordinary skill in the art that such a method employs conventional or modified sonar techniques.

Once the analysis has been performed, an ejection acoustic wave having a focal point at about a cell center near the fluid surface is generated in order to eject at least one droplet of the fluid, wherein the optimum intensity and directionality of the ejection acoustic wave is determined using the aforementioned analysis optionally in combination with additional data. The “optimum” intensity and directionality are generally selected to produce droplets of consistent size and velocity. For example, the desired intensity and directionality of the ejection acoustic wave may be determined by using not only the spatial relationship assessed as above, but also geometric data associated with the reservoir, fluid property data associated with the fluid to be ejected, cell dimensions and consequent cell volume, and/or by using historical cell containing droplet ejection data associated with the ejection sequence. In addition, the data may show the need to reposition the ejector so as to reposition the acoustic radiation generator with respect to the fluid surface, in order to ensure that the focal point of the ejection acoustic wave is near the fluid surface, where desired. For example, if analysis reveals that the acoustic radiation generator is positioned such that the ejection acoustic wave cannot be focused near the fluid surface, the acoustic radiation generator is repositioned using vertical, horizontal and/or rotational movement to allow appropriate focusing of the ejection acoustic wave.

Because one aspect of the invention is ejection of a single cell, the selective nature of the invention will be immediately appreciated. Using simple ejection, cells of sufficiently different size can be separated, starting with ejection of the smallest cells and this can be employed as a type of cell sorter in addition to a method for making arrays. For example because monocytes (D≈20 μm) are much larger than both small (D≈8 μm) and medium and large lymphocytes (D≈12-14μm), corresponding to a cellular volume for monocytes of about 3 times (large lymphocytes) to about 16 times (small lymphocytes) greater a mixture of these cells may be selectively ejected for arraying or sorting. The minimum acoustic energy level adequate to eject small lymphocytes will be insufficient to eject the large lymphocytes that are approximately 5 times as voluminous and massive and monocytes which are approximately 16 times as voluminous and massive.

Once all the small lymphocytes have been ejected the large lymphocytes may be ejected using minimum acoustic energy level adequate to eject large lymphocytes (which will be adequate for ejecting medium lymphocytes) with little danger of ejecting monocytes, which are approximately 3 times as voluminous and massive. Surface functionalization with cognate moieties to marker moieties inherently or by transformation displayed externally on a cell exterior offers another level of selectivity, albeit requiring ejection onto a surface. Finally, as the invention provides for acoustic location of a cell to determine whether it is close enough to the surface to be ejected, various properties may be measured and used as additional criteria for ejection. One of skill in the art of cell sorting will appreciate that such ejection with additional criteria can be adapted to traditional cell sorting applications by ejection in a trajectory appropriate to transfer the ejected cell to another fluidic container, or by spotting onto a substrate and subsequently washing the desired cells into a container as desired.

The ability to measure a property as an ejection criterion, in addition to permitting the invention to be used for cell sorting, permits the sorting of non-living solids, gels and fluid regions discrete from the carrier fluid. It will be readily appreciated that the ejection of, for example, beads used for solid phase combinatorial synthesis and bearing some marker or property identifying the combinatorial sequence may be separated by the method of the invention.

### EXAMPLE 1

Acoustic Ejection of Monocytes Onto a Substrate As An Array

Rabbit polyclonal-Ab against human MHC (displayed on all cells) is generated and a single clone is selected which binds a MHC epitope common to all humans rather than to the epitopes specific to individuals. A substrate is functionalized with the mAb by routine methods, monocrystalline Si is chosen as substrate because of the plethora of known methods for functionalizing Si. A channel having dimensions of 25 μm width and 25 μm depth, and about 3 cm length, open on top for the last 0.5 cm is utilized to economize on time spent searching for cells to eject. The channel is fabricated of an HF etched glass plate heat fused to a cover glass plate by routine microfabrication techniques.

The channel is fluidically connected by routine methods to a fluid column to which the cell suspension is added. The dimensions of the column allow 5 ml of fluid carrier and cells to be added so that a sufficient column pressure exists to initiate fluid flow through the channel to allow fluid to reach the open top area in a sufficiently short time, after which the top of the column is connected to a pressure regulator which allows the gas pressure above the carrier fluid in thee column to be regulated to permit fine adjustment, termination and reinitiation of the carrier fluid flow through the channel.

The carrier fluid may be a physiologic saline or other electrolyte solution having an osmolality about equivalent to that of blood serum. The monocytes are spotted onto a substrate maintained at about 38° C. The substrate employed is planar, and the density of 10,000 sites/cm² is chosen, with each site occupied by a single cell. Circulating monocytes from 10 different individuals are obtained and purified by routine methods.

The monocytes of each individual are attached to the array by acoustic ejection of a droplet having a volume of about 4.2 pL in a pattern. Specifically, every tenth site of each row is spotted with monocytes from one individual, and the deposition of that individual's cells is staggered in subsequent rows to permit more separation between cells from an individual. Separation of an individual's cells is preferable because it provides an internal control against variation in conditioned between different substrate areas. The monocytes from the remaining individuals are spotted onto the array sites in acoustically ejected droplets. Ten duplicate arrays are made.

Because monocytes are attracted by chemotaxis into inflamed tissues where they transformed into macrophages under the influence of immune mediators, the arrays are studied by immersing them in various physiologic solutions containing one or more inflammatory mediators, such as histamine, interleukins (Ils), granulocyte macrophage colony stimulating factor (GM-CSF), leukotrienes and other inflammatory mediators known in the art, as well as conditions which might affect inflammation, such as heat, and known antiinflammatory agents including steroids, non-steroidal antiinflammatory drugs, and random substances or those suspected to affect the activation of macrophages. It will be readily appreciated that certain mediators and combinations thereof will have a pro- or anti-inflammatory effect, and that there will be differences between individuals and to a lesser extent between individual cells. Because the monocytes are attached by the mAb/MHC specific attachment, the array will not be disrupted by immersion.

The transformation of the monocytes into macrophages and of macrophages back to monocytes may be observed by light microscopy without affecting cell viability. Other known methods including EM and XPS (X-ray photoelectron spectroscopy) of individual cells. Because immune cells, especially activated macrophages are able to activate immune cells by release of immune mediators and chemotactic agents, the possibility exists that one individuals monocytes are not responsive to an immune mediator or condition, but responsive to the immune mediators released by another individuals macrophage which was responsive to the experimental condition. To control for the preceding, standard well plates are used as controls using the identical method, with multiple monocytes from the same individual in each well (for 96 well plates, 9 wells/individual, 110 cells each). A final control using well plates without the mAb/MHC attachment system is also created by the method described, surface tension sufficing to hold the ejected cell containing droplets in place, and it is readily appreciated that the 110 droplets deposited in each well plate are preferably deposited at different locations within the well to prevent droplets too big to be held in place by surface tension from being formed by multiple deposition.

### EXAMPLE 2

Human Airway Epithelium (HAE) Cell Array for Studying Airway Immune and Inflammatory Response

The method of the preceding example is adapted to HAE cells by providing a channel having appropriate dimensions (just larger than the HAE cells). Alternatively the width of the channel is just wider than the cells, but to permit faster loading, the depth is approximately three times the diameter of the cells and a ramp as depicted in FIG. 5D is employed in the channel flow path just prior to the channel region which is open. Alternatively a photon field as may be provided by a laser as commonly used in optical tweezers may be employed to force the cells close to the surface. HAE cells may be obtained by routine biopsy and cultured. Before being loaded for ejection they must be suspended as individual cells by disaggregating them by conventional tissue culture methods.

The experiments may be conducted under conditions which do permit cell division. The need for the preceding as well as the conditions required for this will be appreciated by one of ordinary skill. The controls with well plates are useful but not as critical as with the monocytes.

### EXAMPLE 3

HAE Cell Array For Studying Individual Susceptability To Mutagenesis As a Proxy For Carcinogenesis

The method of the preceding example is adapted to permit exposing the arrayed HAE cells to chemical and other mutagens such as heat and radiation. Genetic damage is measured at different times after the exposure is discontinued by routine methods for biochemical assaying of broken crosslinked and otherwise damaged DNA. Differences in DNA repair enzyme genetics may be studied by comparing recovery (extent of reduction of damage) at various times after exposure. The well plate arrays remain useful as controls, and cells may be cultured in the well plates or array cells may be removed and cultured to determine whether there is actual appearance of dysplastic or neoplastic cells in subsequent cell generations after the exposure.

### EXAMPLE 4

Cell Patterning

The method of Examples 1 and 2 is adapted to pattern basal squamous cells. Basal squamous keratinizing epithelial cells and squamous non-keratinizing epithelial cells are patterned on a nitrocellulose substrate functionalized as in Example 1. The pattern generated emulates the vermillion border of the lip. The patterned cells on substrate are then immersed in suitable culture media, and studies for forming a skin/non-keratinizing junction.

### EXAMPLE 5

Acoustic Ejection of Lymphocytes from Blood Onto An Epitope Array

Small, medium and large lymphocytes are ejected by the methods of the preceding examples to form a clonal epitopic array. Two different dimension channels appropriately designed to force the cells near the surface are constructed side by side. The wider channel is about 15 μm wide for medium and large lymphocytes; the narrower channel is 10 μm wide for small lymphocytes. Small lymphocytes may be separated from large and medium lymphocytes by routine methods, or by acoustic ejection. An amount of energy barely sufficient to eject small lymphocytes is applied with all lymphocytes in the mixture passing through one common channel (15 μm wide). The energy is applied to each lymphocyte which is detected at the channel opening or aperture which forms the ejection region. The ejected lymphocytes may be ejected onto a substrate and washed into a petri dish or other container. Alternatively, the acoustic energy can be delivered to eject the droplet in a non-vertical trajectory so that the droplets land in a nearby container, such as a channel that is open on top sufficiently near the ejection channel.

The epitope array is a combinatorial tetrapeptide array formed from naturally occurring amino acids. Other epitopes are readily appreciated to exist both in proteins as a result of nonprimary structure and from peptidic molecules bearing haptens or other biomolecules such as peptidoglycans or polysaccharides. Thus only a small fraction of the approximately 10 ¹² epitopes will be arrayed. Both T and B cells will bind these epitopes, by slightly diferent mechanisms as will be readily appreciated. The tetrapeptide arrays can be made by various methods, for example by adaptation of solid phase peptide synthesis techniques to focused acoustic ejection of reagents as described in the copending application on combinatorial chemistry described above. As 1.6×10 ⁴ different natural tetrapeptides exist, 16 1 cm² array synthesis areas must be made to make all the tetrapeptides and maintain appropriate density for allowing separation of individual cells.

Cells are spotted onto the array sites as rapidly as possible (thus two channels for maintaining single file line of cells in the channels despite the different sizes). When each array site (all 16,000 sites) has had a droplet ejected onto it, the arrays are washed to remove cells that do not bind the epitope at the site of deposition. The arrays are imaged to determine which sites bind a cell, and the cycle is repeated for sites not binding a cell, which are re-spotted. Immediately apprehended is that this process requires imaging of the array after washing, and overall must be automated. Automation of such a system is readily attainable, and invaluable information and clonal separation would be derived prior to completion of the project. Use of different types of epitopes would further extend the cataloguing.

### EXAMPLE 6

Ejection of Bacteria To Select Transformed Bacteria

E. coli are transformed routine methods to express pancytokeratin, a eukaryotic protein, by a construct that also causes expression and display of streptavidin on the cell surface. Using a substrate biotinylated by routine methods, the transformed cells selected by acoustic ejection onto the substrate of the E. coli cells onto the substrate as described in the preceding Examples 1-5. The channel size must be adapted to bacterial dimensions (1 μm) but this is attainable by known microfabrication methods. Transformed cells will be specifically bound to the biotin cognate moiety by the marker moiety, streptavidin. Washing the substrate will remove cells that have not been transformed, leaving only transformed cells attached to the substrate.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains. All patents, patent applications, journal articles and other references cited herein are incorporated by reference in their entireties.

We claim:

1. A method for ejecting a cell from within a fluid near the surface thereof comprising delivering sufficient focused energy to eject the cell contained in a droplet of said fluid.

2. The method of claim 1, wherein said focused energy comprises focused acoustic energy.

3. The method of claim 1, wherein said focused energy comprises focused electromagnetic energy.

4. The method of claim 1, further comprising detecting of whether said cell is sufficiently close to the surface for ejection.

5. The method of claim 1, further comprising detecting of whether said cell possesses a property to select the cell for ejection.

6. The method of claim 1, wherein said ejection is onto a substrate surface.

7. A method for ejecting a cell from a plurality of cells present in a fluid having a fluid surface to a locale on a substrate surface, said method comprising the steps of:
(a) detecting in the fluid a candidate cell;
(b) determining the distance between said cell and the fluid surface; and
(c) delivering sufficient focused energy to eject said candidate cell as a droplet contained cell onto said locale of said substrate surface from said fluid if the distance in (b) is sufficiently small, said droplet contained cell present in a fluid droplet ejected from the fluid.

8. The method of claim 7, wherein said plurality of cells are substantially the same size and said fluid droplet has a sufficiently small volume capable of containing a single cell.

9. The method of claim 7, wherein said plurality of cells may be grouped into at least two different groups, each different group comprising cells of substantially the same size, wherein the different groups differ substantially in mean cell size, whereby said fluid droplet has a sufficiently small volume capable of containing a single cell of the different group having the smallest mean cell size.

10. The method of claim 7, wherein said detecting of step (a) is by acoustic detection of a volume contained in said fluid having a different acoustic impedance than said fluid.

11. The method of claim 7, wherein said detecting of step (a) further comprises determining whether said detected candidate cell possesses a property and said delivering of focused acoustic energy of step (c) requires said candidate cell to possess said property.

12. The method of claim 7, wherein said locale of said substrate surface specifically binds said candidate cell to effect a specific binding, whereby any cell displaying said marker molecule is attached to the substrate surface by the specific binding of said substrate surface to said marker molecule to yield a selective substrate attachment of only those cells displaying said marker molecule.

13. The method of claim 7, further comprising:
(d) exposing the substrate to conditions that remove any cell not displaying said marker molecule, but do not disrupt said selective substrate attachment sufficiently to dislodge any cell displaying said marker molecules.

14. The method of claim 7, wherein said detection is of a cell as a localized volume having a different acoustic impedance than the fluid.

15. The method of claim 7, wherein said focused energy comprises focused acoustic energy.

16. The method of claim 7, wherein said detection is of a cell as a localized volume having a different acoustic impedance than the fluid, and said focused energy comprises focused acoustic energy.

17. The method of claim 7, wherein said detection is of a cell as a localized volume having a different refractive index than the fluid.

18. The method of claim 7, wherein said focused energy comprises focused electromagnetic energy.

19. The method of claim 7, wherein said detection is of a cell as a localized volume having a different refractive index than the fluid, and said focused energy comprises focused electromagnetic energy.

20. A method for separating, from a plurality of cells having an approximately equivalent volume present near a fluid surface, a cell that displays a marker molecule from a cell not displaying said marker molecule, said method comprising the steps of:
(a) detecting in a fluid a cell;
(b) determining the distance between said cell and the fluid surface;
(c) delivering sufficient focused energy to eject said cell onto a substrate surface from said fluid if the distance in (b) between said cell and the fluid surface is sufficiently small for ejection, said cell contained in a fluid droplet ejected from the fluid, said fluid droplet having a sufficiently small volume capable of containing a single cell having said approximately equivalent volume, said substrate surface specifically binding said marker molecule to effect a specific binding, whereby any cell displaying said marker molecule is attached to the substrate surface by the specific binding of said substrate surface to said marker molecule to yield a selective substrate attachment of only those cells displaying said marker molecule; and
(d) exposing the substrate to conditions that remove any cell not displaying said marker molecule, but do not disrupt said selective substrate attachment sufficiently to dislodge any cell displaying said marker molecule.

21. The method of claim 20, wherein said detection is of a cell as a localized volume having a different acoustic impedance than the fluid.

22. The method of claim 20, wherein said focused energy comprises focused acoustic energy.

23. The method of claim 20, wherein said detection is of a cell as a localized volume having a different acoustic impedance than the fluid, and said focused energy comprises focused acoustic energy.

24. The method of claim 20, wherein said detection is of a cell as a localized volume having a different refractive index than the fluid.

25. The method of claim 20, wherein said focused energy comprises focused electromagnetic energy.

26. The method of claim 20, wherein said detection is of a cell as a localized volume having a different refractive index than the fluid, and said focused energy comprises focused electromagnetic energy.

27. The method of claim 20, further comprising, (d) if the distance in (b) is not sufficiently small for ejection in step (c), applying focused energy to move said cell closer to the surface for ejection and repeating step (c).

28. A system for the separation, from a carrier fluid containing a plurality of cells having an approximately equivalent volume present near a fluid surface a cell that displays a marker molecule from a cell not displaying said marker molecule, said system comprising:
a fluidic container;
a substrate having a substrate surface substantially parallel to a plane that contains said fluid surface, said substrate surface specifically binding said marker molecule to effect a specific binding, whereby any cell displaying said marker molecule is attached to the substrate surface by the specific binding of said substrate surface to said marker molecule to yield a selective substrate attachment of only those cells displaying said marker molecule;
an acoustic ejector of fluid droplets onto said substrate surface, comprising an acoustic radiation generator for generating acoustic radiation and a focusing means for focusing the acoustic radiation at a focal point near the fluid surface; and
a means for positioning the ejector relative to said substrate in acoustic coupling relationship to said channel in an appropriate position to permit said focusing means to focus the acoustic radiation at said focal point,
wherein cells present in said carrier fluid that are detected sufficiently near the fluid surface for ejection, are ejected from said carrier fluid in a fluid droplet onto said substrate surface, and said cell displaying said marker molecule is held in place by said specific attachment under conditions removing a cell not displaying said marker molecule.

29. The system of claim 28, wherein said fluidic container comprises a fluidic channel that has an opening on top, said fluidic channel having dimensions permitting the carrier fluid containing said plurality of circumscribed volumes to flow freely through said channel.

30. The system of claim 28, wherein a plurality of different displayed markers are employed and said ejected cells contained in said fluid droplets are targeted to a substrate surface comprising a spatial array of localized sites, each localized site known to specifically bind one of the plurality of different displayed markers, whereby cells having each different displayed markers are specifically attached to different localized sites.

31. A system for the separation, from a carrier fluid having a surface and containing a plurality of circumscribed volumes having a different acoustic impedance than said carrier fluid, of one or more of said circumscribed volumes, said system comprising:
a fluidic channel that has an opening on top, said fluidic channel having dimensions permitting the carrier fluid containing said plurality of circumscribed volumes to flow freely through said channel;
a substrate above said opening having a substrate surface substantially parallel to a plane that contains said opening;
means for acoustically ejecting from said carrier fluid onto a location on the substrate surface a circumscribed volume having a different acoustic impedance than said carrier fluid,
wherein said circumscribed volume present in said carrier fluid that is detected near the fluid surface below said opening may be acoustically ejected from said carrier fluid onto a substrate location in a fluid droplet depending upon whether said localized volume possesses one or more properties.

32. A system for the separation, from a carrier fluid containing a plurality of cells having an approximately equivalent volume present near a fluid surface a cell that displays a marker molecule from a cell not displaying said marker molecule, said system comprising:
a fluidic container;
a substrate having a substrate surface substantially parallel to a plane that contains said fluid surface, said substrate surface specifically binding said marker molecule to effect a specific binding, whereby any cell displaying said marker molecule is attached to the substrate surface by the specific binding of said substrate surface to said marker molecule to yield a selective substrate attachment of only those cells displaying said marker molecule;
an acoustic ejector of fluid droplets onto said substrate surface, comprising an acoustic radiation generator for generating acoustic radiation and a focusing means for focusing the acoustic radiation at a focal point near the fluid surface; and
a means for positioning the ejector relative to said fluidic container in acoustic coupling relationship to said channel in an appropriate position to permit said focusing means to focus the acoustic radiation at said focal point,
wherein cells present in said carrier fluid that are detected sufficiently near the fluid surface for ejection and below said surface, are ejected from said carrier fluid in a fluid droplet onto said substrate surface, and said cell displaying said marker molecule is held in place by said specific attachment under conditions removing a cell not displaying said marker molecule.

33. The system of claim 32, wherein said fluidic container comprises a fluidic channel that has an opening on top, said fluidic channel having dimensions permitting the carrier fluid containing said plurality of circumscribed volumes to flow freely through said channel;

34. The system of claim 32, wherein a plurality of different displayed markers are employed and said ejected cells contained in said fluid droplets are targeted to a substrate surface comprising a spatial array of localized sites, each localized site specifically binding one of the plurality of different displayed markers, whereby cells having each different displayed markers are specifically attached to different localized sites.

35. A system for the separation, from a carrier fluid having a surface and containing a plurality of circumscribed volumes having a different acoustic impedance than said carrier fluid, of one or more of said circumscribed volumes, said system comprising:
a fluidic channel that has an opening on top, said fluidic channel having dimensions permitting the carrier fluid containing said plurality of circumscribed volumes to flow freely through said channel;
a substrate above said opening having a substrate surface substantially parallel to a plane that contains said opening;
means for acoustically ejecting from said carrier fluid through said opening onto a location on the substrate surface a circumscribed volume having a different acoustic impedance than said carrier fluid,
wherein said circumscribed volume present in said carrier fluid that is detected near the fluid surface below said opening may be acoustically ejected from said carrier fluid onto a substrate location in a fluid droplet depending upon whether said localized volume possesses one or more properties.

36. An array of cells on a substrate surface comprising an array of substantially planar sites on said substrates surface, wherein each site contains a single cell.

37. A method for screening an array of individual cells comprised of an array of substantially planar sites, with each site containing a single cell, said method comprising delivering a fluid droplet onto at least one of said single cells contained in each site, said fluid droplet having a volume adequate to immerse said cell in said fluid, said volume being insufficient for said fluid to spread outside of said site.
